(19) United States
(12) Patent Application Publication
(10) Pub. No.: US 2008/0077137 A1
(43) Pub. Date: Mar. 27, 2008

(54) Posterior stabilization for fixed center of rotation anterior prosthesis of the intervertebral disc

(76) Inventor: Richard A. Balderston, Philadelphia, PA (US)

Correspondence Address: WERNER & AXENFELD, LLP, P. O. BOX 1629, WEST CHESTER, PA, 19380, US

(21) Appl. No.: 11/716,961

(22) Filed: Mar. 12, 2007

Related U.S. Application Data
(60) Provisional application No. 60/847,745, filed on Sep. 27, 2006.

(51) Int. Cl. A61B 17/70

(52) U.S. Cl. 606/61

(57) ABSTRACT

A spinal implant assembly used in conjunction with an artificial disc to prevent the artificial disc from disconnecting or shifting after being implanted in a patient. The spinal implant assembly prevents rotation of an artificial disc beyond a maximum range of motion for the artificial disc. That is, the implant assembly prevents the artificial disc from over rotating in any particular direction which could result in a failure to the artificial disc and/or instability to the spine. In its capacity as a safety device, the spinal implant assembly of the invention is passive permitting an artificial disc to rotate without interference or resistance, so long as the range of rotation for the disc remains within safe limits. The spinal implant assembly includes at least one stabilization member attached to vertebrae above and below the artificial disc. The stabilization member is curved and is adapted to compress and expand while retaining its curved shape.

### CROSS-REFERENCE TO RELATED APPLICATIONS

The present patent application claims benefit of U.S. Provisional Application Ser. No. 60/847,745 filed on Sep. 27, 2006. The content of the aforementioned application is fully incorporated by reference herein.

### TECHNICAL FIELD

This invention relates generally to spinal implant assemblies used for implantation into the intervertebral space, and more specifically to a device that provides stabilization of disc replacements that have a fixed-center of rotation.

### BACKGROUND

A human spinal column is highly complex housing and protecting nerves and circulatory elements in close proximity. Bones and connective tissue of the spinal column consist of over twenty bones coupled together in a sequential fashion. Each of the bones are coupled together by a tri-joint system including an anterior (forward) disc, and two posterior (rear) facet joints. The anterior disc is the soft cushioning structure located between the individual bones of the spine, called vertebra. The disc is made of cartilage-like tissue and is flexible enough to allow the spine to bend, curve, and twist in nearly every direction.

Problems with the disc caused by trauma, stress, and degenerative wear, are a few examples of conditions that can result in the need for surgical intervention. Traditionally, the operative treatment for chronic disc pain and other painful spinal conditions has been spinal fusion. This is a surgical procedure in which the disc causing pain is removed and vertebras above/below the disc are fused together. By removing disc tissue and eliminating movement, pain is usually significantly reduced.

A major drawback of spinal fusion, however, is a reduced level of mobility for a patient. Fusion of two bones in the spine, limits the overall flexibility of the spinal column, and artificially restricts normal motion of the patient. This constraint of movement can lead to other problems, such as premature breakdown of adjacent discs, and other collateral stresses.

A newer procedure, known as artificial disc replacement has recently been introduced in the U.S., which avoids fusion and preserves spinal motion. In particular, artificial disc replacement is a medical procedure in which a natural disc in the spine is replaced with an artificial disc, which is designed to mimic the biomechanical action of the natural disc. That is, an artificial disc (also referred to as a disc replacement, disc prosthesis, spine arthroplasty device, and the like) is a device that is implanted into the spine to imitate the functions of a normal disc including carrying loads and permitting motion.

When a total disc replacement is performed, in which all or most of the natural disc is removed, an artificial disc is implanted into the space between the vertebras in place of the removed disc. Such implants may use a ball and socket joint or some other design, which provide a center of rotation to simulate natural movement of a disc. Thus, an artificial disc does not restrict motion and is designed to imitate normal movement between adjacent vertebrae of the spine.

A potential complication associated with an artificial disc replacement is the potential for a movement of the artificial disc after being surgically implanted. That is, the concern with an artificial disc is the potential for a translational, nonconcentric motion between the two vertebrae due to lack of ligamentous support either at the disc annulus level or due to failure of the facet joints and posterior ligamentous structures. This instability may cause the spine to be able to move beyond maximal angle of flexion or translation. Such a shift of the vertebrae may cause the spinal column above the implant to move out of alignment compared to the normal alignment and thus, place the nerves at increased danger for catastrophic compromise of nerve function. movement of the implant may cause vascular, neurological damage, spinal cord impingement or other damage.

Another problem that may result postoperatively, is instability of the spine as a result of the implant being able to rotate beyond a range of motion permitted by the implant.

### SUMMARY

Described herein is a spinal implant assembly for stabilizing an artificial disc replacement that has a fixed-center of rotation. In particular, the spinal implant assembly of the present invention is a safety device used in conjunction with an artificial disc, which prevents rotation of the artificial disc beyond a maximum range of motion for the artificial disc (design limit of the device). That is, the implant assembly prevents the artificial disc from over-rotating in any particular direction which could result in a failure to the artificial disc and/or instability to the spine. In its capacity as a safety device, the spinal implant assembly of the invention is generally passive permitting an artificial disc to rotate without interference or resistance, so long as the range of rotation for the disc remains within safe limits.

In accordance with one embodiment of the invention, a spinal implant assembly includes a stabilization member, attachable to at least one lateral posterior side of a spine. The stabilization member is adapted to have its endpoints secured to vertebrae above and below an artificial disc. The stabilization member is curved in shape forming an arc. The arc of the stabilization member is secured to the vertebrae such that it has a fixed radius with respect to a center of rotation point of the artificial disc. The stabilization member is adapted to compress and expand while retaining its curved shape as well as the fixed radius, when there is either posterior flexion or posterior extension of the spine, respectively. The stabilization member will only permit expansion or compression within range of motion deemed safe for the artificial disc.

In accordance with another embodiment of the invention, stabilizing a spine with a fixed center of rotation prosthesis of an intervertebral disc is achieved, by securing at least one stabilization member to pedicle portions of, discs above and below an artificial disc on at least one lateral side of the posterior side of a spine. The stabilization member is curved in shape in the form of an arc and includes a first endpoint and a second endpoint. As part of the securing process, the stabilization members is adjusted such that a first fixed radius is measured from a center of rotation point of the artificial disc to the first endpoint, and a second fixed radius is measured from the center of rotation point to the second endpoint of the stabilization member. In one embodiment, the stabilization member may be secured to the spine by fastener members, which are attached to a pedicle portion of a spine above and below the artificial disc.

Additional exemplary implementations and features/advantages are described in the Detailed Description in conjunction with the accompanying drawings below. The scope of the invention is recited in the Claims or equivalents thereto.

### BRIEF DESCRIPTION OF THE DRAWINGS

The detailed description is explained with reference to the accompanying figures. In the figures, the left-most digit(s) of a reference number identifies the figure in which the reference number first appears. It should be noted that the figures are not necessarily drawn to scale and are for illustration purposes only.

FIG. 1 shows a side view of two adjacent vertebrae of a spine: a superior vertebra bone and an inferior vertebra bone.

FIG. 2A is a posterior view of the same portion of spine as shown in FIG. 1.

FIG. 2B is identical to FIG. 2A, but shows that the position of each stabilization member is adjustable, such as being placed more in-line with the center of the spine.

FIG. 3 shows a sample flexion motion of a portion of the spine with the stabilization member expanding in length as attachment points X and Y move away from each other.

FIG. 4 shows a sample extension motion of a portion of the spine with the stabilization member contracting in length as attachment points X and Y move toward each other.

FIG. 5 shows a side view of an exemplary fastening member for connecting a stabilization member to the spine.

FIG. 6 shows a cross-sectional side view of an exemplary embodiment for implementing a stabilization member that includes two tubes that partially intussuscept one another.

FIG. 7 shows a cross-sectional axial view of tubes comprising an exemplary implementation of a stabilization member.

FIGS. 8 and 9 show a planar view of another exemplary embodiment for implementing a portion of a stabilization member that includes an expandable rod-type intussusception model.

FIG. 10 is a side view of a portion of a link showing a cross-sectional view of a track with a post from another link connected to the track.

FIG. 11 shows a planar view of and exemplary embodiment of a link having an exemplary connection post at an endpoint of a stabilization member (chain rod configuration) for connecting the stabilization member to fastener members.

### DETAILED DESCRIPTION

Reference herein to “one embodiment”, “an embodiment”, “an implementation” or “one implementation” or similar formulations herein, means that a particular feature, structure, operation, or characteristic described in connection with the embodiment, is included in at least one embodiment of the present invention. Thus, the appearances of such phrases or formulations herein are not necessarily all referring to the same embodiment. Furthermore, various particular features, structures, operations, or characteristics may be combined in any suitable manner in one or more embodiments.

In the following description, for purposes of explanation, specific numbers,’ materials and configurations are set forth in order to provide a thorough understanding of the present invention. However, it will be apparent to one skilled in the art that the present invention may be practiced without each specific example. In other instances, well-known features are omitted or simplified to clarify the description of the exemplary embodiments of the present invention, and thereby, to better explain the present invention.

The inventor intends these embodiments and implementations to serve as representative illustrations and examples. The inventor does not intend these embodiments to limit the scope of the claims including all possible equivalent elements therein; rather, the inventor has contemplated that the claimed invention might also be embodied and implemented in other ways, in conjunction with other present or future technologies.

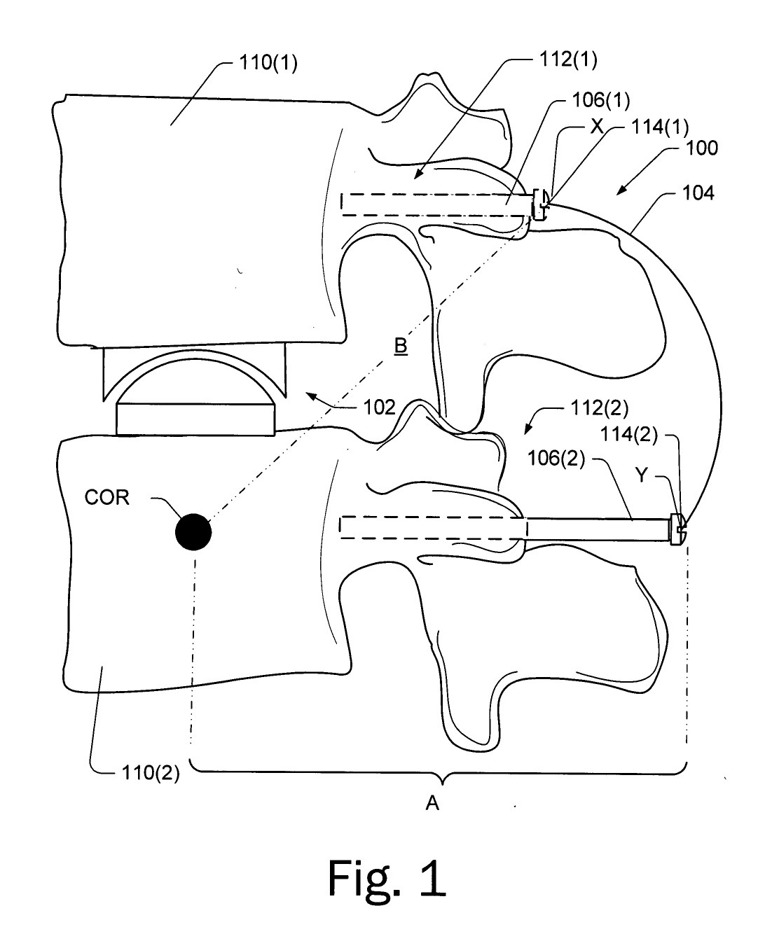

FIG. 1 shows a side view of two adjacent vertebrae of a spine: an upper (superior) vertebra 110(1) and lower (inferior) vertebra 110(2). Implanted between vertebrae 110(1), 110(2), in the anterior portion of the intervertebral space is a fixed center-of-rotation anterior prosthesis (artificial disc) 102. In one embodiment, artificial disc is a total replacement device that provides a centroid of motion centrally located within the intervertebral space, also referred to as a “center-of-rotation (COR)” plane for artificial disc 102.

It should be appreciated by those skilled in the art after having the benefit of this disclosure that artificial disc 102 includes any intervertebral prosthesis device configured to permit free range of motion between two adjacent vertebral bones 110(1), 110(2). Furthermore, artificial disc 102 is not necessarily limited to a particular brand or design configuration, and may include variations in design including less than a total disc replacement configuration. Artificial disc 102 may also include any prosthesis implanted in between any suitable vertebrae of the five regions (cervical, thoracic, lumbar, sacral, and coccygeal) of the spine.

Used in conjunction with artificial disc 102 is a stabilization assembly 100 to prevent rotation of the artificial disc beyond a maximum range of motion for the artificial disc (design limit of the device).

In one embodiment, assembly 100 may include at least one stabilization member 104, which is attached to the pedicle regions 112(1), 112(2) of superior and inferior vertebrae bones 110(1), 110(2). In one embodiment, assembly 100 may also include fastener members 106(1), 106(2) to anchor stabilization member 104 to adjacent vertebrae bones 110.

It should be appreciate by those skilled in the art after having the benefit of this disclosure, that pedicle regions 112 of vertebrae bones 110 provide a stronger boney platform in which to attach stabilization member 104 via fastener members 106, but that it is possible to attach stabilization member 104 to other areas of vertebrae bones 110(1), 110(2) (e.g. pars area) without necessarily departing from the scope of this invention. Further, assembly 100 may be installed to any suitable boney structure or non-boney substrate, above or below the intervertbral space in which an artificial disc 102 is implanted, including any suitable boney structures associated with the posterior side of the five regions of the spine.

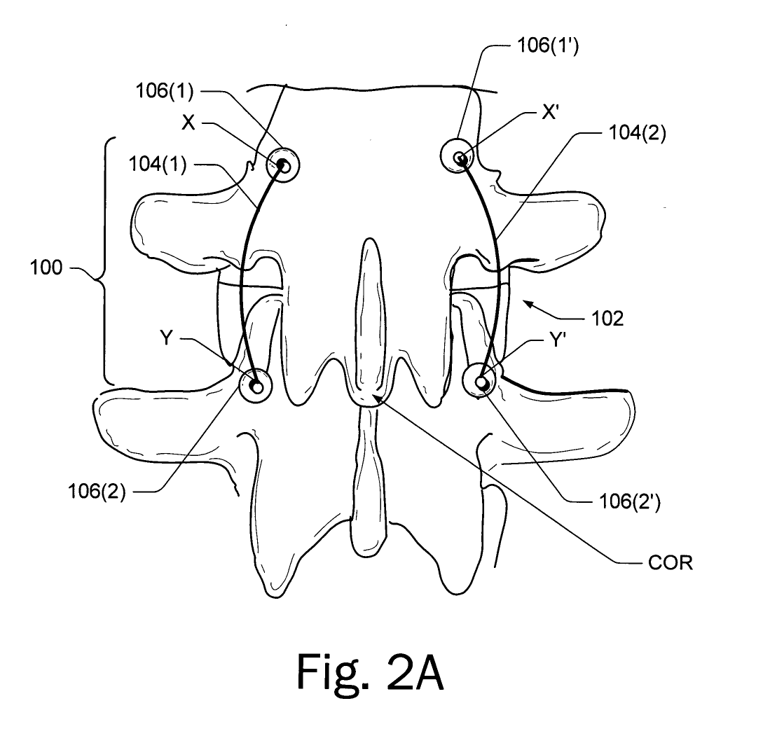

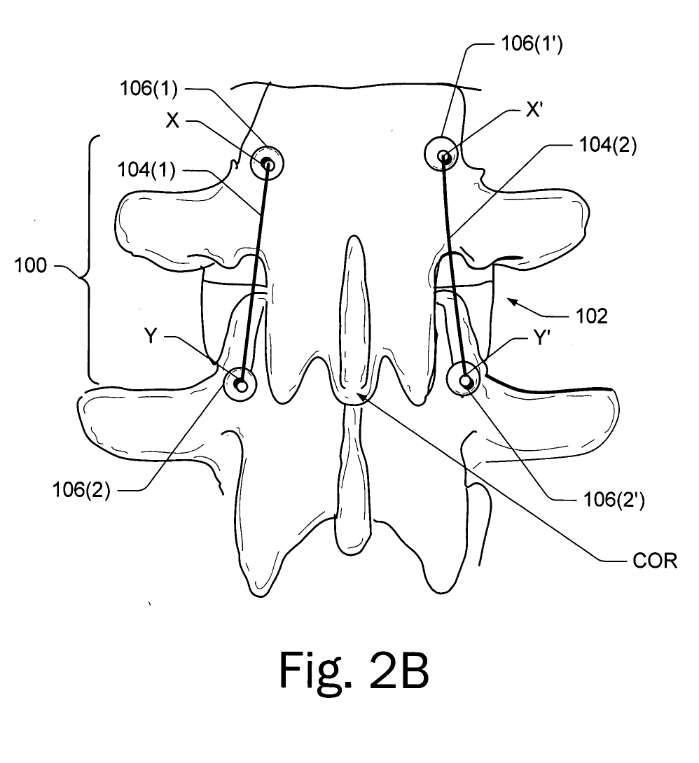

FIG. 2A is a posterior view of the same portion of spine as shown in FIG. 1. FIG. 2A shows that, in another embodiment, assembly 100 may include two stabilization members 104(1), 104(2) attached bilaterally to vertebrae bones 110(1), 110(2). Each bilateral side of assembly 100 includes essentially the same devices as shown in FIG. 1. Although the exemplary assembly 100 and artificial disc 102 illustrated herein are shown for use with two intact vertebrae, it is appreciated that assembly 100 and artificial disc 102 may be attached to, or implanted in, vertebral bones (or substitute structures) that are not fully intact and may have missing anatomical elements or other defects.

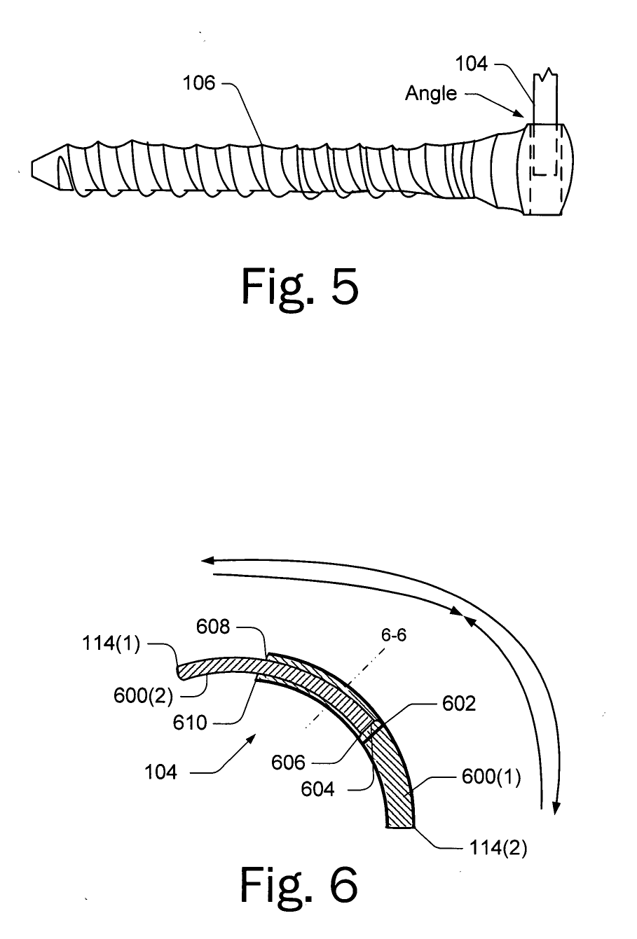

In one embodiment, fastener members, referred to generally as reference number 106, may include pedicle screws (such as shown in FIG. 5) used for anchoring stabilization member 104 into to the pedicle regions 112 of vertebrae bones 110. However, it should be appreciated by those skilled in the art after having the benefit of this disclosure, that any suitable type of anchoring device may be used in place of pedicle screws 106 without departing from the scope of this invention. For instance, in other embodiments other devices may be used to secure stabilization member 104 to the spine, such as, for example, various combinations and sub-combinations of hooks, anchors, prongs, screws, and expandable flanges.

FIG. 2B is identical to FIG. 2A, but shows that the position of each stabilization member is adjustable, such as being placed more in-line with the center of the spine.

Referring back to FIG. 1, stabilization member 104 has two endpoints 114(1), 114(2) for attachment to fastener members 106(1), 106(2), at attachment points X and Y, respectively.

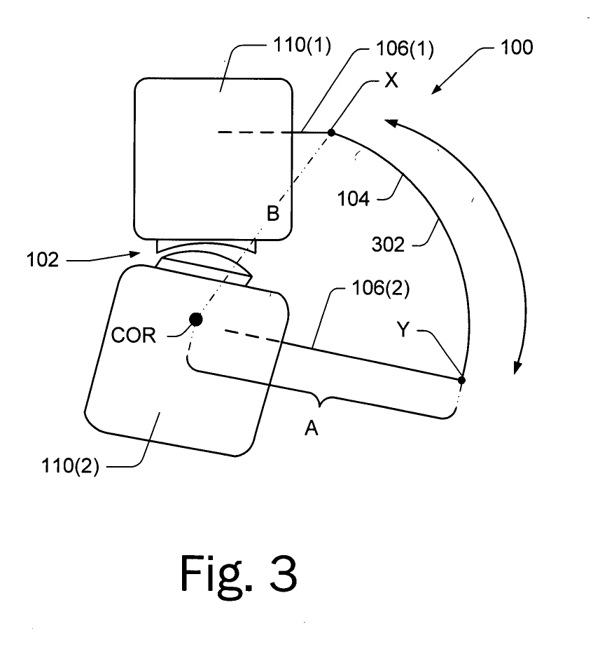

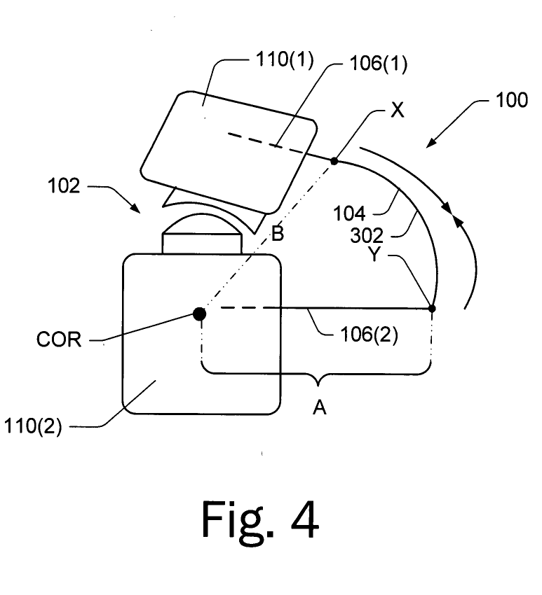

Stabilization member 104 is curved or arc shaped, and is able to compress and expand, but still retain its general curved shape. That is, stabilization member 104 provides an expandable and compressible connection between attachment points X and Y, which are in an arc of motion 302 (FIGS. 3 and 4) relative to the COR for stabilization member 104.

Attachment points X and Y are generally the same distance “A” and “B” from the COR. That is, “A” generally equals “B”, which is the radius of a fixed distance to the curve shape of stabilization member 104 from the COR.

For example, FIG. 3 shows sample flexion motion of the spine with stabilization member 104 expanding in length as attachment points X and Y move away from each other. In this embodiment, stabilization member 104 has a predetermined maximum expansion length due to flexion motion in which the distances between endpoint attachment points “X” and “Y” are furthest from each other. When stabilization member 104 reaches the maximum expansion length, stabilization member 104 will not expand further and therefore it limits (i.e., restricts) posterior flexion motion of the spine by preventing attachment points X and Y from moving beyond a maximum predetermined distance apart. Accordingly, stabilization member 104 prevents any change in distance between point X and the COR so that the superior vertebra bone 110(1) is not free to move off the center axis with respect to the COR or slide off the inferior vertebra bone 110(2).

Likewise, FIG. 4 shows sample extension motion of the spine with stabilization member 104 contracting in length as attachment points X and Y move toward each other. In this embodiment, stabilization member 104 has a predetermined smallest contraction length due to extension motion in which the distances between endpoint attachment points “X” and “Y” are closest to each other. When stabilization member 104 reaches the smallest contraction length, stabilization member 104 will not contract further and therefore it limits (i.e., restricts) posterior extension motion of the spine by preventing attachment points X and Y from moving too close together. Accordingly, stabilization member 104 prevents any change in distance between point Y and the COR so that the inferior vertebra bone 110(2) is not free to move off the center axis with respect to the COR or slide off the superior vertebra bone 110(1).

With reference to FIGS. 3 and 4, distances “A” and “B” from the COR point for artificial disc 102, form a radius which remain constant (e.g., fixed) during either arcs of motion, even as the angle of the spine extends or flexes. In one embodiment, stabilization member 104 permits a total motion of about 11 to 20 degrees along the arc of motion 302 of the stabilization member 104. It should be appreciated by those skilled in the art after having the benefit of this disclosure that the total motion along the arc of motion 302 is adjustable by the surgeon and may vary slightly greater or smaller than the aforementioned range without departing from the scope of this invention.

Additionally, the exact degree and angle for the curvature selected for stabilization member 104 may vary. For example, different sized and shaped stabilization members may be made available and selected by a surgeon before or during surgery based on the size and anatomy of boney structures of a patient. Alternatively, stabilization member 104 may be adjustable in size and shape, such that it can be configured by a surgeon during an operation to provide a desired range of motion. It should also be appreciated by those skilled in the art after having the benefit of this disclosure that the distance for “A” and “B” may be slightly different, and that the functionality provided by stabilization member 104 may be achieved if there are minor tolerable differences between “A” and “B”.

Thus, a surgeon will select length “A” by placing a fastening device 106(2) in the inferior vertebra bone 110(2), and will select length “B” by placing a fastening device 106(1) in the superior vertebra bone 110(1), such that the posterior distal ends of both fastening devices 106(1), 106(2) (at points X and Y) are approximately equal distant from the fixed COR for artificial disc 102.

As stabilization member 104 is curved in shape connecting points X and Y, stabilization member 104 has a sliding mechanism (examples of which will be described) allowing points X and Y to approach or retreat from each other along the arc 302 described by a circle (or some variation of a circular shape) having a radius of “A” or “B” which are generally the same distance.

Having introduced the assembly 100, it is now possible to describe particular exemplary embodiments for implementing stabilization member 104 and attaching it to fastening members 106.

FIG. 5 shows a side view of an exemplary fastening member 106 for connecting stabilization member 104 to the spine. In one embodiment, stabilization member 104 is attached to fastening member 106 at attachment point Y, such that there is a right angle of about 90 degrees between fastening member 106(2) and stabilization member 104. In other words, in one embodiment stabilization member 104 at attachment point Y is generally parallel to an axial plane running through the COR for artificial disc 102. Of course, it is appreciated by those skilled in the art after having benefit of this disclosure that the exact angle may be larger or smaller than 90 degrees, and is not necessarily restricted to 90 degrees. It is also readily apparent to those skilled in the art after having benefit of this disclosure that any suitable attachment mechanism (including other devices) may be used to achieve the desired angle of attachment for coupling stabilization member 104 to fastening member 106.

In one embodiment, stabilization member 104 is attached to fastening member 106 at attachment point X, at a variable angle between fastening member 106(1) and stabilization member 104, which is generally established at surgery with a lateral x-ray of the spine. In one embodiment, a general range of angles for attachment point X is ranges between about 95 and 145 degrees. Again, it is appreciated by those skilled in the art after having benefit of this disclosure how to achieve the desired variable/adjustable angle of attachment for coupling stabilization member 104 to fastening member 106, and that such devices are readily available in the surgical field.

In one embodiment, each fastening member 106 is a pedicle screw having either a generally fixed angle connection head or variable connection head, for attaching the stabilization member at attachment points X and Y.

As explained above, stabilization member 104 includes a sliding mechanism allowing its endpoints to approach or retreat from each other along the arc of motion 302. Stabilization member 104 is adapted to reach a fixed length and to stop compressing when the curvature distance between attachment points X and Y reach a predetermined shortest distance between each other. On the other hand, stabilization member 104 is further adapted to reach a fixed length and to stop expanding when the curvature distance between points X and Y reach a predetermined longest distance between each other. It is noted that in this disclosure endpoints 114(1), 114(2) of stabilization member 104 generally correspond to attachment points X and Y, or are proximal thereto.

FIG. 6 shows a cross-sectional side view of an exemplary embodiment for implementing stabilization member 104. With respect to FIG. 6, exemplary stabilization member 104 includes two subsections: an inner tube 600(1) and an outer tube 600(2). That is an inner tubular member 600(1) is configured to fit and slide inside an outer tubular member 600(2).

Each tube moves in an opposite direction relative to the other tube. For example, as stabilization member 104 contracts (as shown in FIG. 4) with endpoint 114(1) moving toward endpoint 114(2), a portion of inner tube 600(1) slides inside outer tube 600(2) as outer tube 600(2) slides in the opposite direction over inner tube 600(1). Correspondingly, when stabilization member 104 expands (as shown in FIG. 3) with endpoint 114(1) moving away from endpoint 114(2), a portion of inner tube 600(1) slides out of outer tube 600(2), as outer tube slides in the opposite direction.

Tubes 600 may be constructed of a suitable material able to maintain a generally curved state. For example, in one embodiment, each tube is constructed of a rigid material such as stainless steel, or titanium. Alternatively, in other embodiment, one or more of the tubes 600 may be constructed of plastic, rigid rubber, or some composite of such aforementioned materials, or other materials having related properties, as would be appreciated by those skilled in the art having the benefit of this disclosure.

Optionally, friction-reducing material, such as Teflon (polytetrafluoroethylene) or polyamide coating or some other suitable tubing material, is provided on or within an inner surface of outer tube 600(2), and an outer surface of inner tube 600(1), to reduce friction between the two tubes, and thereby facilitate sliding of the tubes relative to each other when stabilization member 104 extends or contracts such as shown in FIGS. 3, 4, and 6.

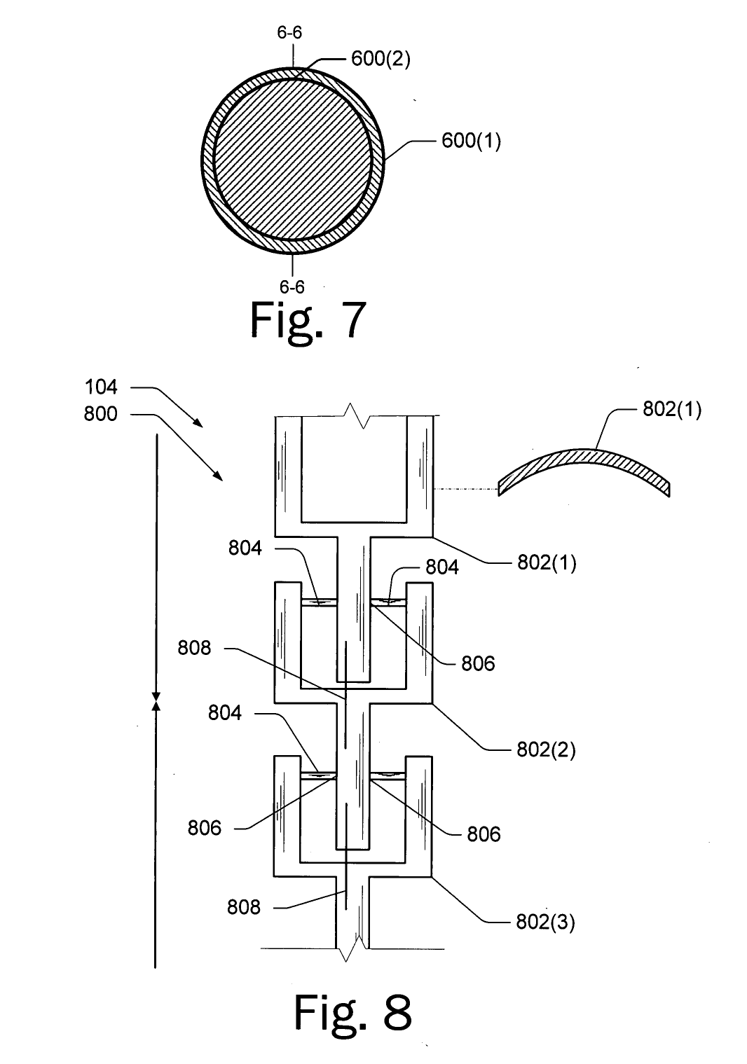

Generally, inner tube 600(1), and outer tube 600(2) are dimensioned to fit closely to each other to present a substantially closed area at distal end 608 of outer tube 600(2), to prevent fluids from entering the inside outer tube 600(2). For example, FIG. 7 shows a cross-sectional axial view of tubes 600(1), and 600(2) comprising an exemplary implementation of stabilization member 104. According to FIG. 7, inner tube 600(1) fits within outer tube 600(2) in a concentric fashion.

Referring back to FIG. 6, stopping mechanisms may be included inside outer tube 600(2) to stop inner tube 600(1) from advancing beyond a certain point within outer tube 600(2) when stabilization member 104 is contracting as the spine reaches a preferred maximum posterior flexion position. For example, in one embodiment a post 602 acts as a blocking member to stop proximal endpoint 604 of inner tube 600(1) from advancing beyond the point of post 602, when stabilization member 104 is contracting.

Correspondingly, a stopping mechanism may also be included inside outer tube 600(2) (or connected to inner tube 600) to stop inner tube 600(1) from completely advancing and sliding out of outer tube 600(2) when stabilization member 104 is expanding while the spine reaches a preferred maximum posterior extension position. For example, in one embodiment, proximal endpoint 604 of inner tube 600(1) may be flared (see 606), and distal end of outer tube 608 may include a narrowing member 610, such as a post or retention ring. Accordingly, if flare 606 of inner tube 600(1) reaches narrowing member 610 as stabilization member is expanding, flare 606 cannot move past narrowing member 610, which prevents stabilization from expanding any further.

It is appreciated that exact locations of where the two tubes stop in either direction may be in other locations than shown in FIG. 6, and that the mechanisms used to stop the tubes from advancing past each other in either contraction or expansion directions may be implemented in other ways. Further, it is appreciated that other mechanisms may be used to connect the two subsections together.

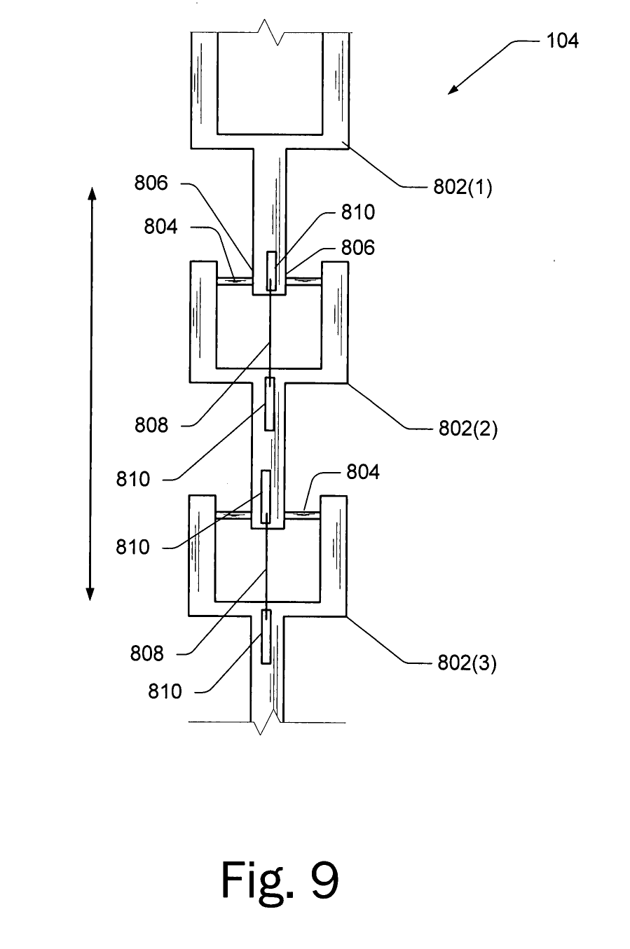

Still, further it is appreciated that stabilization member 104 may be implemented in other ways. For example, in another embodiment, stabilization member 104 includes links of a rod that expand and contract relative to each other. For instance, FIGS. 8 and 9, show a planar view of a portion of a stabilization member 104 implemented as an expandable and compressible rod 800. In particular, FIG. 8 shows stabilization member 104 fully contracted, while FIG. 9 show stabilization 104 fully expanded.

Referring to FIGS. 8 and 9, rod 800 include links 802(1), 802(2), 802(3), . . . , etc. Each link 802 is configured to slide toward or away from an adjacent link via posts 804 that move within a track 806 located on each side of a link 802. Each link 802 may be curved in shape (see the side cross-section view of a link 802(1) in FIG. 8).

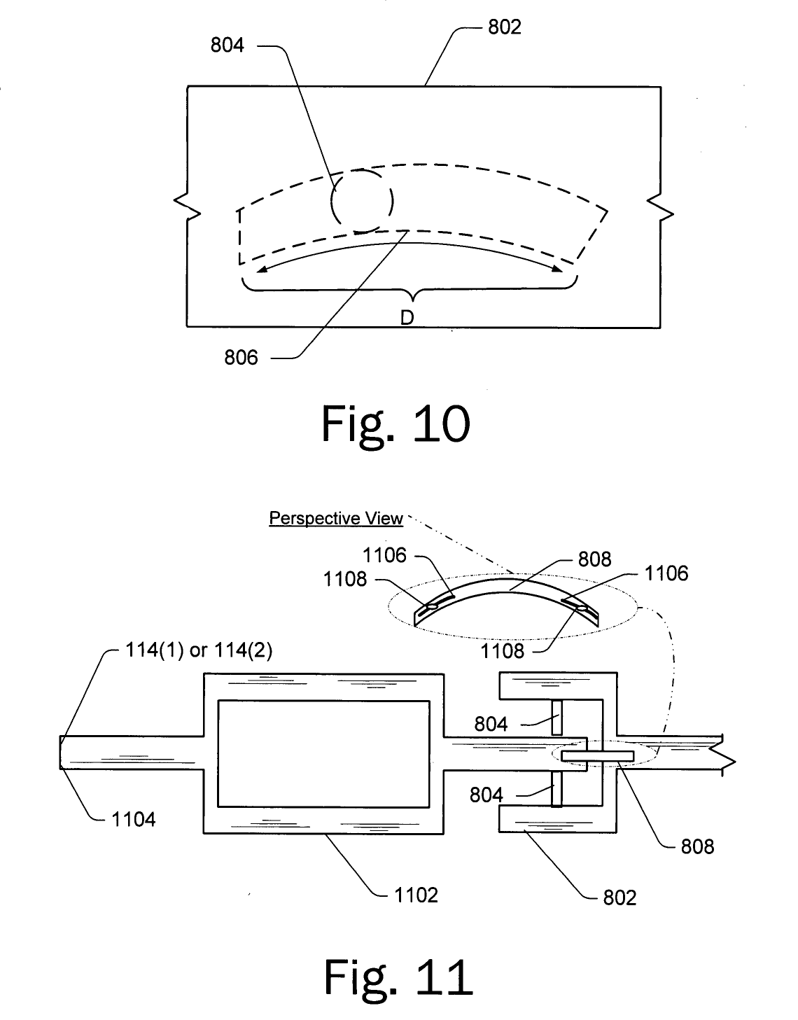

FIG. 10 is a side view of a portion of link 802 showing a cross-sectional view a track 806 in the link with a post 804 from another link engaged in track 806. That is, as shown in FIG. 10, post 804 is configured to move a maximum distance of D in either direction, wherein D is total distance of track 806. This would also permit free slidable movement of each link relative to the other. The collective movement of each link permits overall total movement (contraction and extension) for stabilization member 104. Track 806 may be curved to allow stabilization member 104 to contract or expand while remaining curved in shape.

One or more curved expandable/compressible rigid support members 808 attached to chain 800 may be used to provide curved rigidity to chain 800. Support members 808 may be attached to tracks 810 (FIG. 9) to permit each link to move freely while maintaining an overall fixed curve shape for stabilization member 104. In another embodiment, each rigid support member 808 may have one or more slots 1106 (see FIG. 11) therein that may be attached to each link 802 by a rivet-like member 1108 (FIG. 11) extending from each end of a link. This would also permit free slidable movement of each link relative to the other, while retaining the overall general arc shape of stabilization member 104.

Chain 800 (FIGS. 8 and 9) and its constituent parts, may be constructed of any suitable materials such as stainless steel, titanium, composites, combinations of such materials, or other suitable materials as would be readily appreciate by those skilled in the art, after having the benefit of this disclosure.

FIG. 11 shows a planar view of and exemplary embodiment of a link 802 having an exemplary connection post 1104 at an endpoint 114 of a stabilization member (chain rod configuration 800) for connecting the stabilization member to fastener members. In particular, with reference to FIG. 11, chain 800 may be connected to fastener members 106 by a full link 1102 having a connection post 1104 located at each endpoint 114(1), 114(2) of stabilization member 104.

The exemplary implementation of stabilization member 104 having two curved tubes as shown in FIG. 6, as well as the expandable chain rod device of FIGS. 8-11 are only two exemplary embodiments of a myriad of possible variations for implementing stabilization member 104. Thus, it should be appreciated by those skilled in the art, after having the benefit of this disclosure, that there may be other suitable ways for implementing a stabilization member without departing from this invention.

In conclusion, based on the foregoing, it is anticipated that during an operation one or more stabilization members 104 (regardless of implementation) will be secured to a posterior side of vertebrae above and below an artificial disc on first and second lateral sides (bilaterally) of a spine. Each stabilization member will be curved in shape in the form of an arc having a first endpoint 114(1) (FIGS. 1, 3, 4) and a second endpoint 114(2) (FIGS. 1, 3, 4). Each stabilization member 104 will be adjusted such that a first fixed radius (e.g., X (FIGS. 1, 3, 4)) is measured from a center of rotation (COR) point of the anterior prosthesis (e.g. artificial disc 102) to the first endpoint 114(1), and a second fixed radius Y is measured from the COR point to the second endpoint 114(2) of the stabilization member 104. The process of securing the stabilization member 104 to the spine may include first attaching a pedicle screw 106(1), (FIGS. 1-4) to vertebrae above and a pedicle screw 106(2) (FIGS. 1-4) below the anterior prosthesis, and attaching the first endpoint of the stabilization member to the first pedicle screw, and the second endpoint of the stabilization member to the second pedicle screw. The process of adjusting each stabilization member 104 may include adjusting positioning of the first and second endpoints 114(1), 114(2) (FIGS. 1, 3, 4) relative to the COR point.

Thus, each stabilization member 104 of the invention is attached to the spine and used in conjunction with artificial disc 102 to stabilize the spine. Each stabilization member 104 prevents artificial disc 102 from over-rotating in any particular direction (such as flexion, extension, twisting, left and right lateral bending, and axial rotation or torsion) which could result in a failure to the disc and/or instability to the spine. In its capacity as a safety device, each stabilization member 104 is passive permitting an artificial disc to rotate without interference or resistance, so long as the range of rotation for the disc remains within safe limits.

It is to be understood that the present invention is not limited to the embodiments described above, but encompasses any and all embodiments within the scope of the subjoined Claims including their equivalents.

What is claimed is:

1. An assembly for stabilizing a spine having a fixed center of rotation anterior prosthesis of an intervertebral disc, the assembly comprising:
a first fastener member for attachment to a pedicle portion of a spine above the artificial disc;
a second fastener member for attachment to a pedicle portion of a spine below the artificial disc;
a stabilization member having a first endpoint and a second endpoint, the first endpoint attachable to the first fastener member at an attachment point X, and the second endpoint attachable to the second fastener member at an attachment point Y, wherein the stabilization member is curved in shape, and is adapted to compress and expand, while retaining a generally curved shape, thereby providing an expandable and compressible connection between attachment points X and Y.

2. The assembly as recited in claim 1, wherein the stabilization member is further adapted to reach a fixed length and to stop compressing when the curvature distance between the first endpoint and the second endpoint reaches a predetermined shortest distance between each other.

3. The assembly as recited in claim 1, wherein the stabilization member is further adapted to reach a fixed length and to stop expanding when the curvature distance between the first endpoint and the second endpoint reaches a predetermined longest distance between each other.

4. The assembly as recited in claim 1, wherein when the stabilization member is connected to attachment points X and Y, two fixed distances are formed: a first constant distance is formed measured from a center of rotation point for the artificial disc to the point Y, and a second constant distance is formed measured from the center of rotation point to the point X.

5. The assembly as recited in claim 1, wherein when the stabilization member is connected to attachment points X and Y, two fixed distances are formed: a first constant distance is formed measured from a center of rotation point for the artificial disc to the point Y, and a second constant distance is formed measured from the center of rotation point to the point X, even if the stabilization member compresses and expands with motion of the spine extending or flexing.

6. The assembly as recited in claim 1, further comprising:
a third fastener member for securing to a pedicle portion of a spine above the artificial disc on an opposite lateral side of the spine as the first fastener member;
a fourth fastener member for securing to a pedicle portion of a spine below the artificial disc on an opposite lateral side of the spine as the second fastener member;
a second stabilization member, attachable to the third fastener member at an attachment point X′, and attachable to the fourth fastener member at an attachment point Y′, wherein the second stabilization member is curved in shape, and is adapted to compress and expand, while retaining a generally curved shape, thereby providing an expandable and compressible connection between attachment points X′ and Y′.

7. The assembly as recited in claim 1, wherein the curved shaped of the stabilization member is an arc having a center of rotation point commensurate with the center of rotation of the artificial disc.

8. The assembly as recited in claim 1, wherein the first fastener member is a pedicle screw having a variable angle connection head for attaching the stabilization member at point X.

9. The assembly as recited in claim 1, wherein the second fastener member is a pedicle screw having a generally fixed angle connection head for attaching the stabilization member at point Y such that the stabilization member at point Y is coplanar with a lateral plane of the spine, and perpendicular to an axis of the second fastener member.

10. The assembly as recited in claim 1, wherein the stabilization member is an expandable and compressible rod configured to retain the generally curved shape when expanding or compressing.

11. The assembly as recited in claim 1, wherein the stabilization member is a rod comprising two portions: an inner tubular member configured to fit and slide inside an outer tubular member.

12. The assembly as recited in claim 1, wherein the stabilization member comprises a first tubular member that intussuscepts and is coextensive with at least a portion of a second tubular member, wherein the first tubular member at the first endpoint is configured for attachment to the first fastener member at point X, and the second tubular member at the second endpoint is configured for attachment to the second fastener member at point Y, wherein when points X and Y move toward or away from each other as a result of movement of the spine, this movement causes translational movement of the first tubular member relative to the second tubular member.

13. The assembly as recited in claim 1, wherein the stabilization member comprises a chain having links, each link positioned to slide inward or outward a fixed distance relative to another adjacent link when attachment points X and Y move toward or away from each other, respectively.

14. An assembly for use in conjunction with an artificial disc for securing the artificial disc, the assembly comprising: a first stabilization member attachable to a posterior side of vertebrae on a first lateral side of a spine, the first stabilization member having a first endpoint for securing to a vertebra above and an artificial disc and having second endpoint for securing to a vertebra below the artificial disc, wherein the stabilization member is curved in shape forming an arc having a fixed radius with respect to a center of rotation point for the artificial disc, wherein the stabilization member is adapted to compress and expand while retaining its curved shape and the fixed radius, when there is either posterior flexion and posterior extension of the spine, respectively.

15. The assembly as recited in claim 14, wherein the first stabilization member is further adapted to reach a fixed length and to stop compressing when the distance between the first endpoint and the second endpoint reaches a predetermined shortest distance between each other.

16. The assembly as recited in claim 14, wherein the first stabilization member is further adapted to reach a fixed length and to stop expanding when the curvature distance between the first endpoint and the second endpoint reaches a predetermined longest distance between each other.

17. The assembly as recited in claim 12, further comprising a second stabilization member attachable to a posterior side of vertebrae above and below an artificial disc on a second lateral side of a spine, opposite the first lateral side.

18. The assembly as recited in claim 12, wherein a total length between the first endpoint and the second endpoint of the first stabilization member when either compressed or expanded is adjustable.

19. The assembly as recited in claim 12, wherein the first stabilization member is attachable the posterior side of the spine via pedicle screws.

20. A method for stabilizing a spine having a fixed center of rotation anterior prosthesis of an intervertebral disc, the method comprising:
securing a stabilization member to a posterior side of vertebrae above and below an artificial disc on a first lateral side of a spine, wherein the stabilization member is curved in shape in the form of an arc and wherein the stabilization member includes a first endpoint and a second endpoint; and
adjusting the stabilization member such that a first fixed radius is measured from a center of rotation point of the anterior prosthesis to the first endpoint, and a second fixed radius is measured from the center of rotation point to the second endpoint of the stabilization member.

21. The method as recited in claim 20, wherein securing the stabilization member includes attaching at least first and second pedicle screws to vertebrae above and below the anterior prosthesis, and attaching the first endpoint to the first pedicle screw, and the second endpoint to the second pedicle screw.

22. The method as recited in claim 20, wherein adjusting the stabilization member includes adjusting positioning of the first and second endpoints relative to the center of rotation point.

23. The method as recited in claim 20, further comprising securing a second stabilization member to a posterior side of vertebrae above and below an artificial disc on a second lateral side of a spine opposite the first side, wherein the stabilization member is curved in shape forming an arc; and adjusting the second stabilization member such that a first fixed radius is measured from a center of rotation point of the anterior prosthesis to the first endpoint, and a second fixed radius is measured from the center of rotation point to the second endpoint of the second stabilization member.
